(19) United States
(12) Patent Application Publication
(10) Pub. No.: US 2020/0121570 A1
(43) Pub. Date: Apr. 23, 2020

(54) TEETH WHITENING SYSTEM, COMPOSITIONS AND METHODS

(71) Applicant: JASIBO, LLC, Las Vegas, NV (US)

(72) Inventor: Jennifer Jablow, New York, NY (US)

(21) Appl. No.: 16/166,921

(22) Filed: Oct. 22, 2018

(51) Int. Cl. A61K 8/22; A61Q 11/00; A61K 8/31

(52) U.S. Cl. CPC A61K 8/22; A61Q 11/00; A61K 2800/28; A61K 2800/88; A61K 8/31

(57) ABSTRACT

Composition and methods that include a dual component oral care composition for tooth whitening including a first oral care composition including activated charcoal and a second oral care composition embodiments including an oxidizing agent.

### FIELD

The aspects of the disclosed embodiments generally relate to dental and oral care products, compositions and methods to whiten teeth.

### BACKGROUND

Whiteness of teeth can be an important cosmetic desired element. Common teeth-bleaching method can include oxidizing agents such as hydrogen peroxide, which can be used in a liquid form, and which is mixed with an anhydrous carrier containing glycerin and/or propylene glycol and/or polyethylene glycol. Another oxidizing agent is carbamide peroxide (urea hydrogen peroxide) which can be dissolved in ethanol and which upon contact with water dissociates into urea and hydrogen peroxide. Hydrogen peroxide in the presence of water dissociates into water and an oxygen free radical, the latter being highly reactive and, as a result, react with the stain molecules on teeth making them water soluble and/or transparent or both.

Tooth brushing using activated charcoal in a powder or paste/gel form is technically not “whitening” because it just removes surface stains and does not oxidize organic stains below the tooth surface. As a result, a consumer can be disappointed with the negligible of lack of shade change of their teeth upon use. Use of such products are also leave residue in the mouth, such as, for example, black particles being left in between teeth that can be arduous to remove, as well as staining the bathroom sink where one usually brushes one' teeth. Furthermore, oral or dental use of pure charcoal powder can be very abrasive causing enamel wear, acrylic dental restoration wear and possible tooth sensitivity.

Dental pellicle, or acquired pellicle, is a protein film that forms on the surface enamel by selective binding of glycoproteins from saliva that prevents continuous deposition of salivary calcium phosphate. It can form in seconds after a tooth is cleaned or after chewing. Dental pellicle can also be a physical barrier to tooth whitening compositions penetrating the tooth surface.

It would be advantageous to have a tooth whitening oxidation below the tooth surface with less mess for the consumer.

### BRIEF DESCRIPTION

In one embodiment, a dual component oral care composition for tooth whitening is provided. The dual component oral care composition for tooth whitening includes a first oral care composition including activated charcoal and a second oral care composition including an oxidizing agent.

In another embodiment, a method of whitening teeth is provided. The method of whitening teeth includes applying to a tooth a dual component oral care composition including a first oral care composition including activated charcoal and a second oral care composition including an oxidizing agent.

In another embodiment, a dual component oral care composition for tooth whitening is provided. The dual component oral care composition for tooth whitening includes a first oral care composition having a pH ranging from about 6 to about 9 and including activated charcoal in an amount of from about 0.5 weight % to about 90 weight % and a second oral care composition including hydrogen peroxide in an amount of from about 0.5 weight % to about 35 weight %.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention. As used herein, “about” may be understood by persons of ordinary skill in the art and can vary to some extent depending upon the context in which it is used. If there are uses of the term which are not clear to persons of ordinary skill in the art, given the context in which it is used, “about” may mean up to plus or minus 10% of the particular term.

Compositions and methods of the present disclosure include embodiments, and the use thereof, having a dual component oral care composition for tooth whitening with a first oral care composition embodiments including activated charcoal and a second oral care composition embodiments including an oxidizing agent, such as for example, peroxide or its derivatives. Methods of use embodiments of the present disclosure include simultaneously using the first oral care composition embodiments and the second oral care composition embodiments or sequentially using them in which the first oral care composition embodiments are used first followed by use of the second oral care composition embodiments.

The embodiments of the present disclosure are believed to adsorb surface stains of the tooth and/or remove or disrupt the dental pellicle by using the first oral care composition and, thereby allowing the oxidative material in the second oral care composition to better penetrate the tooth surface to reach and oxidize stains not only on the surface, but below the tooth surface as well, thereby resulting in an improved tooth whitening composition and method of use.

The oral care compositions of the present disclosure can include oral rinses (e.g. mouthrinses or washes), pastes, dentifrices, and oral gels. Oral care compositions include products which, in the ordinary course of usage, are not intentionally swallowed for purposes of systemic administration of particular therapeutic agents, but is rather retained in the oral cavity or the tissues thereof for a time sufficient to be effective for purposes of therapeutic activity within the oral cavity and surfaces and tissues therein, including but not limited to teeth whitening. Teeth, as used herein, refers to natural teeth as well as artificial teeth or dental prosthesis. Oral cavity includes teeth, tissues (including mucous membranes and cheek tissue in the oral cavity) and the surfaces thereof present in mouth.

Embodiments of the present disclosure are dual component oral care compositions including first oral care composition embodiments having pharmaceutically acceptable activated charcoal alone (e.g., activated charcoal powder) or contained in an orally acceptable vehicle, preferably in the form of a paste or gel orally acceptable vehicle, more preferably in the form of a paste orally acceptable vehicle that is homogeneous and semi-solid in form.

As used herein, “activated charcoal” and “activated carbon” (which can be used interchangeably) refer to a form of carbon in particulate or powder form that has been processed to make it extremely porous. Activated charcoal thus possesses a very large surface area, which provides a very important adsorption capacity. Charcoal becomes activated charcoal when the charcoal is subjected to high temperatures combined with a gas or activating agent to increase its porosity and expand its surface area. Activated charcoal is a porous material that can adsorb impurities from its surrounding environment which is different than absorption. Through adsorption, the porous surface of activated charcoal attracts (mostly unwanted) material into its pores. This leaves the area around the activated cleaner and, when present in the mouth, therefore can remove stains and debris from a tooth surface. Charcoal may have a useful catalytic ability for the decomposition of peroxides, such as H₂O₂. Therefore, mixing the carbon or charcoal with a peroxide source may catalyze the decomposition of peroxide resulting in faster oxidization of organic stains on the tooth surface and inside the tooth structure.

The activated charcoal may be derived from at least one material selected from the group consisting of wood, wood dust, wood flour, bamboo, cotton linters, peat, coal, coconut (including, e.g., coconut shell), lignite, carbohydrates, petroleum pitch, petroleum coke, coal tar pitch, fruit pits, fruit stones, nut shells, nut pits, sawdust, palm, vegetables (e.g., eggplant), synthetic polymer, natural polymer, lignocellulosic material, and combinations thereof, preferably from coconut shell, bamboo or eggplant because of the high purity of the activated charcoal from those sources. The activated charcoal can be employed at concentrations of from about 0.5 weight % to about 90 weight %, preferably from about 2 weight % to about 40 weight %. The activated charcoal is preferably powdered or fine granular activated charcoal with a particle size less than 1.0 mm in size with an average diameter ranging from about 0.15 to about 0.25 mm. Although any pore size for the activated charcoal can be used including micropores (<2 nm in diameter), mesopores (2-50 nm in diameter) and macropores (>50 nm in diameter), the differences in the size of their width openings being a representation of the pore distance, activated charcoal including macropores are preferable because the larger pore size results in better adsorption. Preferable activated charcoal includes activated charcoal with a higher total pore volume, for example, provide by coconut shell-based activated carbons, because of the increased macroporosity enhanced adsorption in which a larger pore size can result in greater NOM (natural organic matter) adsorption by the activated charcoal particle.

The first oral care composition embodiments can have a pH of above about 5.8, preferably from about 6 to about 9. The higher pH may help neutralize acid in the mouth and catalyze decomposition of the peroxide in the second oral care composition embodiments.

Other components of the first oral care composition embodiments can include one or more of the following: a preservative, a binder, a humectant, a flavoring agent, a chelating agent, a sweetener, an occlusive agent (to prevent or retard water loss of a composition to which it is part), an anti-bad-breath agent, an opacifying agent (to make a composition more opaque), a viscosity modifier or thickener, an abrasive, a fluoride source, an agent used to diminish teeth sensitivity, a remineralizing agent and a surfactant.

An oral or pharmaceutically acceptable preservative can include one or a mixture of preservatives, such as, for example, benzoic acid, sodium benzoate, methylparaben, propylparaben, sorbic acid and potassium sorbate. These preservative agents are generally present at levels ranging from about 0.01 weight % to about 2 weight %. For the first oral care composition embodiments, the preferred preservative is sodium benzoate.

Embodiments of the present disclosure can also include sources of fluoride ions, or fluorine-providing compounds useful, for example, as anti-caries agents known for their ability to release fluoride ions in water. An oral or pharmaceutically acceptable fluoride source can include one or a mixture of fluoride source, such as, for example, fluoride salts, such as soluble alkali metal and alkaline earth metal salts, for example, sodium fluoride, potassium fluoride, ammonium fluoride, calcium fluoride, copper fluorides such as cuprous fluoride, zinc fluoride, barium fluoride, sodium fluorosilicate, ammonium fluorosilicate, sodium fluorozirconate, sodium monofluoro-phosphate, aluminum mono- and di-fluorophosphate, and fluorinated sodium calcium pyrophosphate. Alkali metal and tin fluorides, such as sodium and stannous fluoride, sodium monofluorophosphate, and mixtures thereof, are preferred. It is preferred to have the fluoride source in the first oral care composition at concentrations ranging from about 0.005 weight % to about 3 weight %.

An oral or pharmaceutically acceptable abrasive or polishing agent can include one or a mixture of abrasives such as for example, calcium carbonate, sodium metaphosphate, potassium metaphosphate, tricalcium phosphate, dihydrated dicalcium phosphate, calcined alumina and siliceous materials, bentonite clay, kaolin clay or combinations thereof. Preferred abrasives include calcium carbonate, siliceous materials, such as silica and more preferably hydrated silica, bentonite clay, kaolin clay or combinations thereof. More preferably abrasives include a mixture of calcium carbonate, bentonite clay, kaolin clay and hydrated silica. The abrasive content the first oral care composition embodiments can be in the range of from about of 1 weight % to about 80 weight % and preferably about 35 weight % to about 50 weight %.

An oral or pharmaceutically acceptable anti-bad-breath agent which are suitable for treating bad breath (e.g. halitosis). can include one or a mixture of anti-bad-breath agents such as for example, cetylpyridinium chloride (CPC), zinc compounds (e.g., zinc chloride, zinc citrate and Zinc gluconate), and chlorhexidine, preferably Zinc Chloride. The anti-bad-breath agents content the first oral care composition embodiments can be in the range of from about of 0.05 weight % to about 5 weight % and preferably about 0.075 weight % to about 2 weight %.

An oral or pharmaceutically acceptable thickener can include one or a mixture of organic and inorganic thickeners or viscosity modifiers. Organic thickeners such as natural and synthetic gums and colloids may also be incorporated in the present invention. Examples of such organic thickeners include carrageenan (Irish moss), xanthan gum and sodium carboxymethyl cellulose, starch, polyvinylpyrrolidone, hydroxyethylpropyl cellulose, hydroxybutyl methyl cellulose, hydroxypropylmethyl cellulose, and hydroxyethyl cellulose. Inorganic thickeners such as Laponite D are preferred, as well as amorphous silica compounds which function as thickening agents including, colloidal silica compounds available under tradenames such as Cab-o-sil fumed silica manufactured by Cabot Corporation and distributed by Lenape Chemical, Bound Brook, N.J., Zeodent 165 from J. M. Huber Chemicals Division, Havre de Grace, Md. 21078 and Sylox 15 from Grace Davison, Baltimore, Md. 21203.

An oral or pharmaceutically acceptable binder, that can also act a viscosity increasing agent, can include one or a mixture of binders, such as for example, water soluble binders, such as, for example polyvinyl alcohols, polyvinyl pyrrolidone (PVP), polyvinyl pyrrolidone derivates and copolymers, saccharides such as D-glucose, D-fructose, sorbitol, polysaccharides such as starch, cellulose and pectin including derivatives thereof, gelatine, naturally occurring substances such as alginic acid, carrageen gum, lucost bean gum, guar gum, xanthan gum, tragacanth gum, arabic gum, karaya gum. Preferably, for the first oral care composition, polyvinyl pyrrolidone (PVP) as well as cellulose gum or xanthan gum can be used as a binder and/or thickener. The binder/thickener content the first oral care composition embodiments may be present in the first oral care composition embodiments in concentrations of about 0.1 weight % to about 10 weight %, preferably about 5 weight % to about 8 weight %.

An oral or pharmaceutically acceptable chelating agent can include one or a mixture of chelating agents, such as for example, sodium tripolyphosphate, ethylenediamine tetracetic acid (“EDTA”) and its salts (e.g., tetrasodium EDTA and calcium EDTA), or a combination thereof. For the aspects of the disclosed embodiments, such chelating agents can be employed at concentrations of from about 0.3 weight % to about 30 weight %. The preferred chelating agent for the first oral care composition embodiments is sodium tripolyphosphate.

An oral or pharmaceutically acceptable occlusive agent can include one or a mixture of occlusive agents, such as, for example, petrolatum, mineral oil, beeswax, silicone oil, lanolin and oil-soluble lanolin derivatives, saturated and unsaturated fatty alcohols such as behenyl alcohol, hydrocarbons such as squalane, and various animal and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil. The occlusive agent content the first oral care composition embodiments can be in the range of from about of 1 weight % to about 35 weight % and preferably about 1 weight % to about 10 weight %. coconut oil is the preferred occlusive agent for the first oral composition.

Embodiments of the present disclosure are dual component oral care compositions including second oral care composition embodiments having a pharmaceutically acceptable oxidizing agent in an orally acceptable vehicle, preferably in the form of a paste or gel orally acceptable vehicle, more preferably in the form of a gel orally acceptable vehicle.

Other components of the second oral or dental composition embodiments can include one or more of the following: a gelling agent, a humectant, a flavoring agent, a bleaching agent/peroxide stabilizer, agent to diminish tooth sensitivity, a chelating agent and a sweetener.

The first and second oral care compositions need to be kept separated prior to use is because some components of the first oral care composition are not compatible during storage with components of the second oral care composition (i.e., the oxidizing agent, e.g., peroxide) because their interaction can lead to reaction with or degradation of some of the components of the first oral care composition as well as affect the integrity/physical state of the compositions including changing the form of the of the first oral care composition (e,g., gel or semi-solid to liquid or other form,). For example, a peroxide in embodiments of the second oral care composition is not compatible with abrasives and metal salts in embodiments of the first oral care composition. Therefore, the first and second oral care composition embodiments need to be physically separated prior to use, e.g., during storage, in separate containers (e.g., a tube or other suitable tooth paste or gel dispenser) so that each retains their respective semi-solid paste or gel state and do not react with one another until placed into contact with each other.

An oral or pharmaceutically acceptable oxidizing agent can include one or a mixture of oxidizing agents including peroxide compounds or its derivatives, such as, for example, hydrogen peroxide, peroxydiphosphate, urea peroxide, metal peroxides such as calcium peroxide, sodium peroxide, stronthium peroxide, magnesium peroxide, and the salts of perborate, persilicate, perphosphate and percarbonate such as sodium perborate, potassium persilicate and sodium percarbonate. The oxidizing agent content the second oral care composition embodiments can be in the range of from about of 1 weight % to about 35 weight % and preferably about 5 weight % to about 14 weight %.

The preferred peroxide for the second oral care composition embodiments is hydrogen peroxide. Preferably, hydrogen peroxide can be employed at concentrations of from about 0.5 weight % to about 35 weight %, preferably about 5 to 14 weight %.

An oral or pharmaceutically acceptable vehicle can be an aqueous vehicle including water as a solvent. For the first oral care composition, water can be present at a concentration of from about 20 weight % to about 70 weight %, preferably from about 40 weight % to about 50 weight %. For the second oral care composition, water can be present at a concentration of from about 30 weight % to about 75 weight %, preferably from about 51 weight % to about 72 weight %.

An oral or pharmaceutically acceptable bleaching agent or peroxide stabilizer can include one or a mixture of such agents, such as, for example, ethylenediamineteraacetic acid, disodium salt; ethylenediamineteraacetic acid, tetrasodium salt; ethylenediaminetetraacetic acid, calcium disodium salt; etidronic acid; citric acid; gluconic acid; sodium citrate; sodium gluconate; sodium phosphate; disodium phosphate; trisodium phosphate; tetrapotassium pyrophosphate; sodium tripolyphosphate as well as sodium stannate and potassium stannate (tin can be a or peroxide stabilizer). The peroxide stabilizer content the second oral composition embodiments can be in the range of from about of 0.01 weight % to about 2 weight % and preferably about 0.05 weight % to about 0.3 weight %.

An oral or pharmaceutically acceptable gelling agent are semisolid, suspension-type systems that can be single-phase gels containing organic macromolecules distributed substantially uniformly throughout the carrier liquid, which is typically aqueous, but also, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol® trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof. Gelling agents can also act as emulsion stabilizers such as carbomers. Preferably, the second oral care composition includes carbomers. The gelling agent content can be in the range of from about of 1 weight % to about 15 weight % and preferably about 2 weight % to about 4 weight %.

An oral or pharmaceutically acceptable surfactant can include one or a mixture of surfactants, such as, for example, certain nonionic, anionic and amphoteric surfactants and can include sulfate, sulfonate and phosphate ester surfactants (e.g., alkyl sulfonates having 10 to 18 carbon atoms and sulfates of monoglycerides of fatty acids having 10 to 18 carbon atoms) as well as salts and derivatives thereof including, for example, sodium lauryl sulfate (SLS) or sodium lauryl ether sulfate (SLES) as well as anionic taurate surfactants including, for example, sodium methyl cocoyl taurate and sodium methyl oleoyl taurate, PEG caster oils, and PEG hydrogenated caster oils, including, for example, PEG-60 Hydrogenated Castor Oil Preferably, the first oral care composition embodiments include sodium lauryl sulfate (SLS) and sodium methyl cocoyl taurate, more preferably, a mixture of sodium lauryl sulfate (SLS) and sodium methyl cocoyl taurate. Oil Preferably, the second oral care composition includes PEG-60 Hydrogenated Castor Oil. The surfactant content can be in the range of from about of 0.1 weight % to about 10 weight % and preferably about 2 weight % to about 5 weight %.

An oral or pharmaceutically acceptable chelating agent can include one or a mixture of chelating agents, such as for example, sodium tripolyphosphate, ethylenediamine tetracetic acid (“EDTA”) and its salts (e.g., tetrasodium EDTA and calcium EDTA), or a combination thereof. For the aspects of the disclosed embodiments, such chelating agents can be employed at concentrations of from about 0.3 weight % to about 30 weight %. The preferred chelating agent for the first oral care composition embodiments is sodium tripolyphosphate and EDTA for the second oral care composition.

An oral or pharmaceutically acceptable humectant can include one or a mixture of humectants, such as, for example, glycerin, sorbitol and polyethylene glycol, preferably, a mixture of glycerin and sorbitol for the first oral care composition embodiments and preferably propylene glycol and glycerin for the second oral care composition embodiments. The humectant content the first oral care composition embodiments and the second oral second composition embodiments can be in the range of from about of 1 weight % to about 30 weight % and preferably about 2 weight % to about 25 weight %.

An oral or pharmaceutically acceptable flavoring agent can include one or a mixture of flavoring agents, such as for example bubble gum flavor, cherry flavor, grape flavor, anise oil, cassia oil, vanilla extract, vanilla creme, orange flavor, anethole, licorice, spearmint oil, phenylacetaldehyde diisobutyl acetal, and mixtures thereof, preferably spearmint essential oil. Some flavoring agents can also act as sweeteners and can be use as such and include, for example, neohespiridin dehydrochalcone, xylitol, Sucralose, and mixtures thereof, preferably xylitol. The flavoring agent content the first oral care composition embodiments and the second oral care composition embodiments can be in the range of from about of 0.05 weight % to about 3 weight % and preferably about 0.5 weight % to about 1 weight %.

An oral or pharmaceutically acceptable agent used to diminish teeth sensitivity in either the first or second oral care composition embodiments can include one or a mixture of such agents, such as, for example, strontium chloride, potassium nitrate and potassium citrate. The agent used to diminish teeth sensitivity content can be in the range of from about of 0.1 weight % to about 5 weight %. A preferred such agent in the second oral care composition embodiments is potassium nitrate.

An oral or pharmaceutically acceptable pH adjuster can include one or a mixture of such agents, such as, for example, sodium bicarbonate, sodium hydroxide, ammonium hydroxide, calcium glycerophosphate, triethanolamine (an organic compound composed of a tri-alcohol & an amine) or a combination thereof. For the aspects of the disclosed embodiments, sodium bicarbonate can be employed at concentrations of from about 5 weight % to about 99.5 weight %, preferably from about 10 weight % to about 80 weight %, except when it is in powder form where it is preferably from about 90 weight % to about 99.5 weight %. For a combination of sodium bicarbonate and sodium hydroxide, sodium bicarbonate can be employed at concentrations preferably from about 15 weight % to about 79 weight % and sodium hydroxide can be employed at concentrations preferably from about 1 to about 5%.

Calcium glycerophosphate is also a dental agent is capable of remineralizing enamel. In certain embodiments of the present disclosure, such a remineralizing agent can also include a phosphate compound, a calcium compound, a calcium phosphate compound, hydroxyapatite and a caseinate. The phosphate compound can include, for example, a monobasic phosphate compound, a dibasic phosphate compound, a tribasic phosphate compound, calcium glycerophosphate, and combinations thereof.

Embodiments of the present disclosure also include a method of using The dual component oral care composition embodiment to, e.g., whiten teeth, by applying to the tooth surface using, e.g., a tooth brush or other acceptable mean, either simultaneously on the same application device or applying the first oral care composition embodiments and the second oral care composition embodiments or by applying the first oral care composition embodiments first followed by the second oral care composition embodiments, the second oral care composition embodiments being applied about 5 minutes or less after use of the first oral care composition embodiments, preferably, within a time range of about 2 minutes to about 5 minutes after use of the first oral care composition embodiments. Other than a tooth brush, other acceptable means of applying the first oral care composition can include, for example, paint on brush, in a strip or as a slurry by finger application. Other than a tooth brush, other acceptable means of applying the second oral care composition can include, for example, gel in tray form; adhesive strips form; gel painted on form; a dual layer strip in which two compositions mix upon application to form an oxidizing agent, for example, peroxide; placing a peroxide gel and a carbon containing gel in a battery operated silicone mouth tray that warms the contents up to 45 degrees Celsius and may have sonic vibration and using the mouth tray to apply the contents of the tray to the teeth; and adding peroxide to a mouth tray that heats up, may have sonic vibration and may have carbon embedded into the tray such that when the carbon is heated, infrared radiation is given off.

The first and second oral care compositions administered to the teeth together can be administered for a period of time ranging from about 30 seconds to about 3 minutes, preferably about 2 minutes brushing. When the first oral care composition is administered first, it can be administered for a period of time ranging from about 1 minute to about 3 minutes preferably about 2 minutes brushing. When the second oral care composition is administered after the first oral care composition, it can be administered for a period of time ranging from about 1 minute to about 30 minutes preferably about 15 minutes, preferably, when performing the method twice a day.

Other embodiments of the present disclosure include a method of applying the first oral care composition embodiments to teeth to allow for a whitening agent, e.g. a peroxide whitening agent such as the second oral care composition embodiment to oxidize organic stains on the tooth surface and inside the tooth structure. Other embodiments of the present disclosure include a method of catalyzing or accelerating the whitening process of a peroxide whitening agent such as the second oral care composition embodiment by applying the first oral care composition embodiments to teeth prior to or simultaneously with the peroxide whitening agent. Other embodiments of the present disclosure include a method of adsorbing surface stains of the tooth and/or removing or disrupting the dental pellicle by applying the first oral care composition embodiments to teeth prior to or simultaneously with the peroxide whitening agent.

Other embodiments of the present disclosure include packing in separate containers the first oral care composition embodiments and the second oral care container embodiments. Other embodiments of the present disclosure include a kit including separate containers of the first oral care composition embodiments and the second oral care container embodiments and may also include instructions for use.

Other embodiments of the present disclosure include a method of brushing with the first oral care composition and then painting on second oral care composition and letting the latter sit on the tooth surface for a period of time ranging from about 5 minutes to about 30 minutes preferably about 15 minutes, preferably, when performing the method once a day. Other embodiments of the present disclosure include brushing with a pure powder form charcoal and peroxide on same toothbrush or brushing with a charcoal powder first then applying peroxide gel immediately after and allowing the peroxide to remain for a period of time ranging from about 1 minutes to about 10 minutes, preferably about 2 minutes. Other embodiments of the present disclosure include brushing with a charcoal powder first and the paint peroxide gel on thereafter and allowing the peroxide gel to sit for a period of time ranging from about 5 minutes to about 30 minutes preferably about 15 minutes. Other embodiments of the present disclosure include brushing with a charcoal paste or powder first and then placing a peroxide strip or patch on the teeth and allowing the peroxide strip or patch to sit for a period of time ranging from about 5 minutes to about 60 minutes preferably about 15 minutes.

Example 1—Water (solvent—less than about 50 weight %); Sodium benzoate (preservative—less than about 1 weight %); PVP (binder—less than about 5 weight %); Sorbitol (humectant—less than about 25 weight %); Sucralose (flavor agent—less than about 1 weight %); Sodium tripolyphosphate (chelating agent—less than about 5 weight %); Xylitol (sweetener—less than about 5 weight %); Coconut oil (occlusive agent—less than about 1 weight %); Glycerin (humectant—less than about 25 weight %); Calcium glycerophosphate (remineralizing agent & pH adjuster—less than about 5 weight %); Titanium dioxide (opacifying agent—less than about 5 weight %); Cellulose gum or xanthan gum (viscosity increasing agent—less than about 5 weight %); Hydrated silica or calcium carbonate (mechanical stain removal—abrasive & viscosity increasing agent—less than about 50 weight %); Sodium methyl cocoyl taurate (surfactant—less than about 1 weight %); Sodium lauryl sulfate (surfactant—less than about 1 weight %); Activated charcoal powder (adsorbent—less than about 1 weight %); Bentonite or kaolin clay or combination (mechanical stain removal—abrasive—less than about 1 weight %); Zinc chloride (an anti-bad-breath agent—less than about 1 weight %); Flavor (—less than about 5 weight %)

Example 2—Water (solvent), Hydrogen Peroxide (oxidizing agent), Xylitol (sweetener), Glycerin (humectant), Carbomer (emulsifier), PEG-60 Hydrogenated Castor Oil (surfactant), Ammonium Hydroxide (pH adjuster), Flavor, Etidronic Acid (bleaching agent or peroxide stabilizer), Potassium Stannate (bleaching agent or peroxide stabilizer).

Example 3—Propylene Glycol (humectant—about 20%-about 40%); (purified) water (solvent—about 20 weight % to about 40 weight %), Glycerin (humectant—about 20 weight % to about 40 weight %), Hydrogen Peroxide (oxidizing agent—about 14 weight %), triethanolamine (pH adjuster—about 1 weight % to about 10 weight %), Carbomer (emulsifier—about 1 weight % to about 10 weight %), Xylitol (sweetener—about 1 weight %), Spearmint Essential Oil (flavor—less than about 1 weight %), Potassium Nitrate (sensitivity reducer—less than about 1 weight %)

This written description uses examples as part of the disclosure, including the best mode, and also to enable any person skilled in the art to practice the disclosed implementations, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A dual component oral care composition for tooth whitening, comprising:
a. a first oral care composition including activated charcoal; and
b. a second oral care composition including an oxidizing agent.

2. The dual component oral care composition of claim 1, wherein the oxidizing agent is peroxide or its derivatives.

3. The dual component oral care composition of claim 1, wherein the first oral care composition is a tooth paste.

4. The dual component oral care composition of claim 1, wherein the second oral care composition is a gel.

5. The dual component oral care composition of claim 1, wherein the activated charcoal is present in the first oral care composition in an amount of from about 0.5 weight % to about 90 weight %.

6. The dual component oral care composition of claim 1, wherein the first oral care composition has a pH ranging from about 6 to about 9.

7. The dual component oral care composition of claim 2, wherein the peroxide in the second oral care composition is hydrogen peroxide which is present in an amount of from about 0.5 weight % to about 35 weight %.

8. The dual component oral care composition of claim 1, wherein the first oral care composition includes an abrasive.

9. The dual component oral care composition of claim 8, wherein the abrasive includes hydrated silica, calcium, bentonite, kaolin clay or a combination thereof.

10. A method of whitening teeth, comprising:
applying to a tooth a dual component oral care composition, including:
a. a first oral care composition including activated charcoal; and
b. a second oral care composition including an oxidizing agent.

11. The method of claim 10, wherein the first oral composition and the second oral composition are simultaneously applied to the tooth.

12. The method of claim 10, wherein the first oral composition and the second oral composition are sequentially applied to the tooth such that the first oral composition is applied and the second oral composition is applied thereafter.

13. The method of claim 11, wherein the dual component oral care composition is applied using a tooth brush.

14. The method of claim 13, wherein the dual component oral care composition is applied for a time period ranging from about 30 seconds to about 3 minutes.

15. The method of claim 12, wherein the first oral care composition is applied by brushing the teeth with the first oral care composition for a period of time ranging from about 1 minute to about 3 minutes and the second oral care composition is applied for a period of time ranging from about 1 minute to about 30 minutes.

16. The method of claim 15, wherein, the second oral care composition is applied within a time range of about 2 minutes to about 5 minutes after the first oral care composition is applied.

17. A kit comprising the dual component oral care composition of claim 1, wherein the first oral care composition and the second oral care composition are in separate containers.

18. A dual component oral care composition for tooth whitening, comprising:
a. a first oral care composition having a pH ranging from about 6 to about 9 and including activated charcoal in an amount of from about 0.5 weight % to about 90 weight %; and
b. a second oral care composition including hydrogen peroxide in an amount of from about 0.5 weight % to about 35 weight %.

19. The dual component oral care composition of claim 18, wherein the first oral care composition includes an abrasive in an amount of from about 1 weight % to about 80 weight %.

20. The dual component oral care composition of claim 19, wherein the abrasive includes hydrated silica, calcium, bentonite, kaolin clay or a combination thereof.
